(19) United States
(12) Patent Application Publication
(10) Pub. No.: US 2026/0257040 A1
(43) Pub. Date: Sep. 3, 2026

(54) ROTATABLE HANDLE FOR MULTI-LUMEN CATHETER

(71) Applicant: SCP ALTIS, Monaco (MC)

(72) Inventors: Emmanouil I. PROTONOTARIOS, Afidnes (GR); Debra Mae KOZAK, Forest Lake, MN (US); Eric AYAZYAN, The Hague (NL)

(21) Appl. No.: 19/582,326

(22) PCT Filed: Oct. 25, 2024
(86) PCT No.: PCT/IB2024/060543

§ 371 Date: Apr. 27, 2026

Related U.S. Application Data
(60) Provisional application No. 63/593,588, filed on Oct. 27, 2023.

(51) Int. Cl. A61M 25/01; A61M 25/00

(52) U.S. Cl. CPC A61M 25/0136; A61M 25/0026; A61M 2025/0037

(57) ABSTRACT

A multiple-lumen catheter apparatus includes an elongated body and a directional control handle that is capable of maintaining multiple fluidic pathways through the handle to lumens in the catheter body while rotating the elongated body about its longitudinal axis. At least one of the non-axial fluidic pathways through the handle can remain stationary during catheter body rotation.

### FIELD OF THE INVENTION

The present invention relates to catheters generally, and more particularly to directional control handles for multiple lumen catheters that are arranged for transporting a fluid therethrough.

### BACKGROUND OF THE INVENTION

The use of catheters for various medical procedures is well known, and typically involves an elongated catheter body that is designed to establish a channel through which surgical instruments and/or fluids may pass to arrive at a targeted location in vivo. In some cases, guiding the distal end of the catheter toward and to the targeted location can be challenging. Common approaches for guiding the catheter body include the use of guide wires visualized under fluoroscopy, and steerable catheters having distal ends that may be physically manipulated by pull wires or other devices accessible to the catheter operator at the proximal end of the catheter.

These guide systems often employ a control handle attached to the proximal end of the catheter body, wherein the control handle may be manipulated by the operator to desirably position and orient the catheter body. Some catheter systems may involve multiple distinct lumens within the catheter body, with at least one lumen dedicated to the transport of one or more fluids. An example of such catheter systems is a fluid thrombectomy catheter, which transports one or more fluids through the catheter to a designated site within the patient's body to establish a suction force that either performs or assists in the thrombectomy procedure, and to carry excised tissue out from the body through the catheter. In some versions, thrombectomy catheters may include at least two separate fluid-carrying lumens, one for fluid delivery, and another for fluid return. Often, such thrombectomy catheters also include a separate lumen for a guide wire, and/or for other implements useful in the procedure.

Optimal positioning and orientation of catheters at or near a targeted location may require rotation about the catheter axis. Rotational adjustment of catheter orientation may be helpful to optimize or enable camera or other procedural instrument access to the procedure site. In the case of thrombectomy catheters, suction ports in the catheter body may need to be oriented in a specific manner to perform as intended, and may, in some embodiments, need to be rotated about the catheter axis during the thrombectomy procedure. Rotation of the catheter body about its longitudinal axis is driven from the proximal end of the catheter body, typically by a control handle. For catheter systems involving multiple lumens, particularly those adapted for fluid transport, tubing connecting fluid sources or destinations to the catheter lumens can become tangled and even kinked as a result of catheter rotation. The risk of fluid flow disruption caused by tube kinking can limit the extent to which catheter operators are able to successfully orient and re-orient the catheter body in situ.

There is therefore a need for catheter systems that permit catheter body rotation without risk of fluid flow disruption at the proximal end of the catheter, and more specifically at the junction between the catheter body and the control handle.

There is also a need to facilitate catheter body rotation about its elongated axis from an ergonomic and multi-function control handle.

### SUMMARY OF THE INVENTION

By means of the present invention, a multiple-lumen catheter may be rotated in situ while maintaining fluidic connections to at least two distinct fluidic paths extending proximally from the catheter body, and without adjusting the relative orientations of the fluidic paths adjacent to the proximal end of the catheter body. In doing so, any tubing defining such fluidic paths is not at risk of tangling or kinking. Rotation of the catheter body is controlled by a directional control handle attached to the proximal end of the catheter body. The directional control handle has a first portion that is rotatable relative to a second portion, wherein the first and second portions correspondingly control rotation of the catheter body relative to at least one of the flow paths.

In one embodiment, a multiple-lumen catheter apparatus includes a catheter body having a distal end, a proximal end, a longitudinal axis extending centrally within the catheter body between the proximal end and the distal end, and a plurality of distinct lumens extending within the catheter body between the proximal and distal ends. The catheter apparatus of this embodiment further includes a directional control handle connected to the proximal end of the catheter body. The directional control handle includes a housing having a first portion and a second portion, and a manifold apparatus adapted to connect to the proximal end of the catheter body. The manifold apparatus includes a junction member and a first connector that is rotatably connected to the junction member about a first axis. The first connector defines at least a portion of a first channel along the first axis for fluidically connecting a first port of the directional control handle to the proximal end of the catheter body. The junction member defines a second channel along a second axis for fluidically connecting a second port of the directional control handle to the proximal end of the catheter body. The control handle may further include a third port that is arranged for fluidic connection to the proximal end of the catheter body through at least one of the first and second channels. The first portion of the housing may be fixedly secured to the first connector and rotatable relative to the second portion of the housing about the first axis.

In some embodiments, the junction member includes a first leg and a second leg, with the first connector being rotatably connected to the first leg. In some embodiments, the catheter apparatus includes a second connector rotatably connected to the second leg of the junction member about the first axis, wherein the second connector defines a portion of the first channel.

In some embodiments, the second channel intersects the first channel at a junction. The first and third ports may be fluidically connected to the first channel through the second connector.

A first tube may fluidically connect the first port to a first lumen of the catheter body. A second tube may fluidically connect the third port to a second lumen of the catheter body. A third lumen of the catheter body may be fluidically connected to the second port through the second channel.

In some embodiments, the second connector may be fixedly connected to the first portion of the housing, and the junction member may be fixedly connected to the second portion of the housing. The portion of the first channel defined by the second connector may be fluidically disconnected from the second channel.

In some embodiments, the first portion of the housing includes a rotation control element that is connectable with a rotation limiter of the second portion for limiting relative rotation of the first and second housing portions. Rotation of the first connector about the first axis may correspondingly rotate the proximal end of the catheter body about the first axis.

A method for controlling a catheter device includes rotating a first portion of a control handle with respect to a second portion of the control handle about a first axis to correspondingly rotate a catheter body about its longitudinal axis, wherein the control handle is connected to a proximal end of the catheter body. The catheter device includes the catheter body having a plurality of distinct lumens extending between the proximal end and a distal end. The control handle includes at least two fluid channels, with a first fluid channel formed along a first axis through a first portion of the control handle and fluidically connected to a first lumen of the catheter body. A second fluid channel is formed along a second axis through a second portion of the control handle, and is fluidically connected to a second lumen of the catheter body, wherein the second axis is non-parallel to the first axis. The first and second axes are capable of being mutually stationary during the rotation of the first portion of the control handle with respect to the second portion of the control handle about the first axis.

A directional control handle for a multiple-lumen catheter device includes a receiver for coupling to a proximal end of the catheter device, and a first portion defining an axial channel along an axis, the first portion including a first port fluidically connected to the proximal end of the catheter device through the axial channel. A second portion of the directional handle includes a second port, and defines a second channel in fluid communication with and extending non-axially from the axial channel for fluidically connecting the second port to the proximal end of the catheter device. The first portion may be rotatable about the axis relative to the second portion. A third port may be associated with one of the first and second portions, and fluidically connected to the proximal end of the catheter device through at least one of the axial channel and the second channel.

In some embodiments, the second port may be fluidically connected to the proximal end of the catheter device through the second channel and the axial channel.

In some embodiments, the third port may be fluidically connected to the proximal end of the catheter device through only the axial channel. The directional control handle may further include a first tube extending between the first port and a first lumen of the catheter device, and a second tube extending between the third port and a second lumen of the catheter device.

### BRIEF DESCRIPTION OF THE DRAWINGS

FIG. 1 is a schematic illustration of a catheter apparatus of the present invention.

FIG. 2 is a cross-sectional view taken along cut line 2-2 of FIG. 1.

FIG. 3 is an enlarged schematic illustration of a portion of the catheter apparatus illustrated in FIG. 1.

FIG. 4 is a schematic cross-sectional view of the portion of the catheter apparatus illustrated in FIG. 3.

FIG. 5 is a schematic view of a portion of the catheter apparatus illustrated in FIG. 3.

FIG. 6 is a schematic partial cut-away view of a portion of a catheter apparatus of the present invention.

FIG. 7A is a schematic view of a portion of a catheter apparatus of the present invention in a first relative rotation condition.

FIG. 7B is a schematic view of a portion of a catheter apparatus of the present invention in a second relative rotation condition.

FIG. 7C is a schematic view of a portion of a catheter apparatus of the present invention in a third relative rotation condition.

### DETAILED DESCRIPTION OF THE INVENTION

Embodiments of the invention will now be described with reference to the attached drawing figures, which are intended to be representative of various possible embodiments consistent with the invention. It is to be understood that other embodiments not illustrated in the accompanying drawing figures or not specifically described herein are nevertheless considered to be within the scope of the present disclosure.

Spatially relative terms, such as “upper”, “lower”, “inner”, “outer”, “above”, below”, and the like may be used herein for ease of description to describe one element or feature in relation to another element or feature, as illustrated in the drawing figures. Such terms, therefore, are not intended to be limiting as to the arrangement or orientation of elements or features of the present invention.

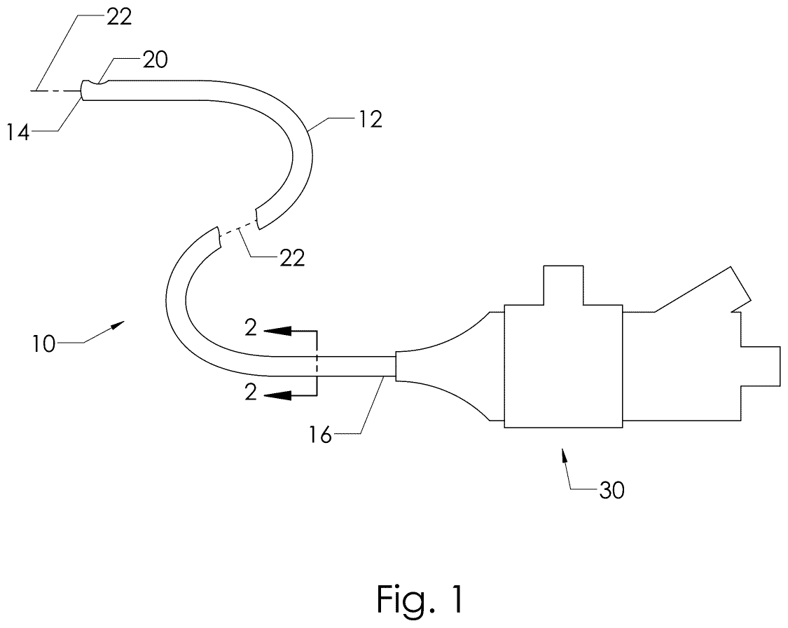

As shown in FIG. 1, a catheter apparatus 10 is arranged for introduction into a body for performing a diagnostic or therapeutic procedure. In some embodiments, catheter apparatus 10 may be arranged for introduction into a body lumen, and may be in the form of an endovascular device. In various embodiments, catheter apparatus 10 may be a guide catheter, a sheath, a procedure catheter, a balloon catheter, or the like. In one embodiment, catheter apparatus 10 may be a thrombectomy catheter for performing hydrodynamic thrombectomy procedures upon a target tissue in vivo.

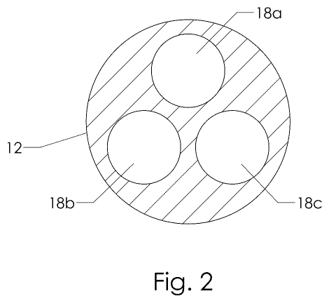

Catheter apparatus 10 may include an elongated tubular body 12 having a distal end 14 and a proximal end 16, and, as illustrated in the cross-sectional view of FIG. 2, one or more lumens 18a, 18b, 18c extending between proximal end 16 and distal end 14. Lumens 18 may be sized and configured as necessary to accommodate passage of instruments, guide wires, optical fibers, tubes, fluids, and the like. In an example embodiment of catheter apparatus 10 being a thrombectomy catheter, first lumen 18a may be configured for accommodating passage of a guide wire (not shown), second lumen 18b may be configured for accommodating passage of a first fluid such as a physician-specified fluid, and third lumen 18c may be configured for accommodating passage of a second fluid such as a return fluid. In some embodiments, second and third lumens 18b, 18c may fluidically communicate with an opening 20 at or near distal end 14 of tubular body 12.

Lumens 18 of catheter apparatus 10 may have a variety of cross-sectional shapes and sizes, and may be consistent or may vary in shape along their length. Lumens 18 may have cross-sectional shapes that are circular, elliptical, oval, rectangular, triangular, and the like.

Body 12 may define a longitudinal axis 22 that extends throughout a length of body 12 and is centrally located at a radial origin along body 12. As will be described in greater detail hereinbelow, body 12 may be rotated about longitudinal axis 22, primarily for appropriately guiding and positioning distal end 14.

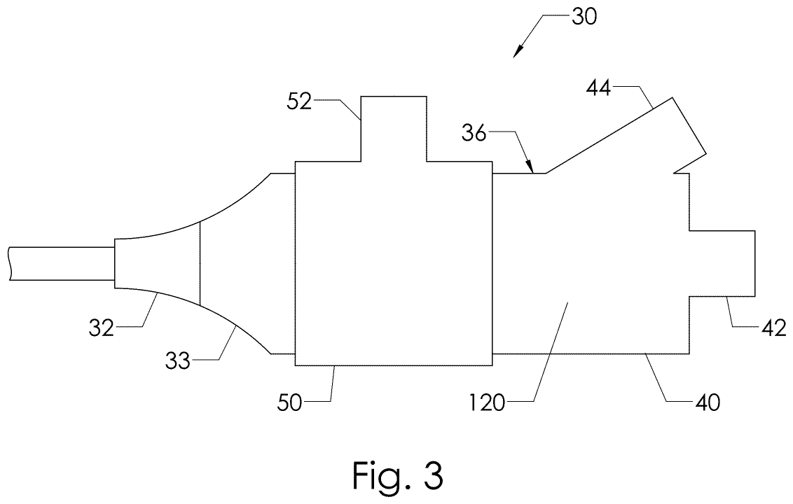

A directional control handle 30 may be connected to catheter body 12 for manipulating body 12, as well as to facilitate fluidic connection between lumens 18 and respective origins/destinations for the various instruments and fluids employed by catheter apparatus 10. As illustrated in FIG. 3, handle 30 includes a receiver 32 for coupling to proximal end 16 of catheter body 12, a first portion 40, and a second portion 50. In some embodiments, handle 30 includes a housing 36 comprising receiver 32, first portion 40, and second portion 50. Receiver 32 may include a strain relief element 33 for reducing strain imparted upon catheter body 12 at the junction between handle 30 and catheter body 12, particularly during manipulation of catheter body 12 by handle 30. Strain relief elements 33 are known in the art of catheter systems.

First portion 40 of housing 36 may include a first port 42 defining a first opening through a wall of housing 36. In some embodiments, first portion of housing 36 may include a second port 44 defining a second opening through a wall of housing 36. In some embodiments, one or both of first and second ports may be positioned proximally with respect to a connection between handle 30 and catheter body 12. In the illustrated embodiment, second portion 50 of housing 36 includes a third port 52 defining a third opening through a wall of housing 36. It is to be understood that first and second portions 40, 50 of housing 36 may comprise distinct walls that together establish housing 30. As described herein, first and second portions 40, 50 of housing 36 may be rotatable relative to one another. In some embodiments, first and second portions 40, 50 of housing 36 are not in contact with one another

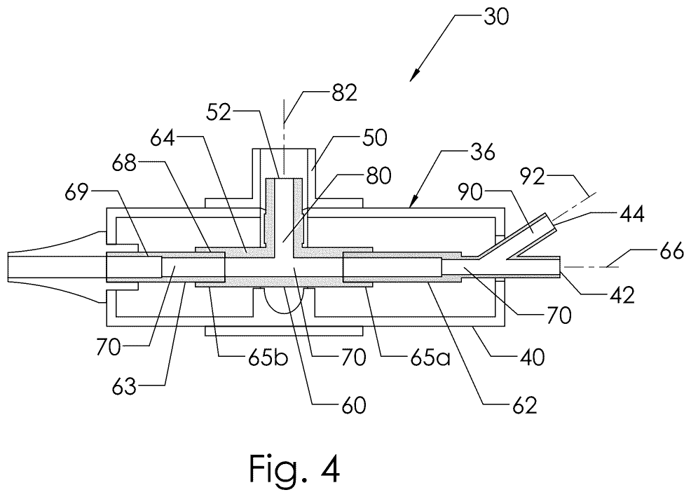

As shown in the cross-sectional view of handle 30 in FIG. 4, handle 30 may include a manifold apparatus 60. In some embodiments, one or more portions of manifold apparatus 60 are connected to one or more portions of housing 36. A first connector 62 of manifold apparatus 60 may be rotatably connected to junction member 64 about a first axis 66. Although a variety of mechanisms are contemplated for facilitating relative rotation among first connector 62 and junction member 64, a bearing 68 may be positioned between first connector 62 and a first leg 65a of junction member 64. Bearing 68 may be any useful bearing type, including a ring bearing, for facilitating relative rotation among junction member 64 and first connector 62. In other embodiments, relative rotation among junction member 64 and first connector 62 may be facilitated without a bearing or a bushing, and may instead be facilitated with one or more relatively low friction materials, such as various plastics. In the illustrated embodiment, first connector 62 may be positioned radially inwardly from first leg 65a of junction member 64. In other embodiments, however, first connector 62 may be positioned radially outwardly of first leg 65a of junction member 64.

In some embodiments, first connector 62 defines at least a portion of a first channel 70. In the illustrated embodiment, first channel 70 extends along first axis 66 for fluidically connecting first port 42 to one or more lumens 18 of catheter apparatus 10 at proximal end 16 of body 12. In the illustrated embodiment, junction member 64 also defines a portion of first channel 70 along first axis 66. First channel 70 may preferably be permanently open between first connector 62 and junction member 64. However, it is contemplated that valves or other selective closure devices may be employed to selectively close and open a fluidic connection of first channel 70 between first connector 62 and junction member 64.

Junction member 64 preferably further defines a second channel 80 extending along a second axis 82 that is not coincident with first axis 66, but may intersect with first axis 66. Second channel 80 may preferably fluidically connect port 52 of second portion 50 to one or more lumens 18 of catheter apparatus 10 at proximal end 16 of body 12. In some embodiments, first channel 70 may intersect with second channel 80, such that port 52 of second portion 50 is may be fluidically connected to one or more lumens 18 through second channel 80 and first channel 70.

In the illustrated embodiment, second port 44 of first portion 40 may be fluidically connected to one or more lumens 18 of catheter apparatus 10 at proximal end 16 of body 12. The fluidic connection may be established through a stem channel 90 that merges with first channel 70 in first connector 62, wherein port 44 is therefore fluidically connected to one or more lumens 18 through first channel 70. Stem channel 90 may include a third axis 92. In some embodiments, third axis 92 is not parallel with first axis 66, but may intersect with first axis 66. It is to be understood, however, that first portion 40 may include only a single port associated with first channel 70, and second portion 50 may include one or more ports associated with second channel 80.

In some embodiments, manifold apparatus 60 may include a second connector 63 rotatably connected to junction member 64 about first axis 66 in a similar manner as first connector 62. In the illustrated embodiment, second connector 63 may be rotatably connected to second leg 65b of junction member 64. Second connector 63 may define a portion of first channel 70, wherein first channel 70, in the illustrated embodiment, extends through first connector 62, junction member 64, and second connector 63 to fluidically connect one or more ports 42, 44, 52 to one or more lumens 18 in catheter body 18.

First connector 62 of manifold apparatus 60 may preferably be fixedly secured to first body 40, such that rotation of first body 40 about first axis 66 correspondingly rotates first connector 62 about first axis 66. In some embodiments, second connector 63 of manifold apparatus 60 is also fixedly secured to first body 40, such that rotation of first body 40 about first axis 66 correspondingly rotates second connector 63 about first axis 66. Junction member 64 of manifold apparatus 60 may preferably be fixedly secured to second body 50, such that rotation of first body 40 relative to second body 50 results in rotation of at least one of first and second connectors 62, 63 relative to junction member 64 about first axis 66.

Preferably, one of first body 40 and second body 50 is secured, directly or indirectly, to catheter body 18, such that control handle 30 may be manipulated to cause rotation of at least proximal end 16 of catheter body 12 with respect to the other of first body 40 and second body 50. In the illustrated embodiment, catheter body 18 may be fixedly secured within a receiver body 69 that may be integrally formed with or secured to manifold apparatus 60. In some embodiments, receiver body 60 may be integrally formed with or secured to one or more of first and second connectors 62, 63, or integrally formed with or secured to junction body 64.

In some embodiments, catheter body may be secured to receiver 32, and one of first and second bodies 40, 50 may be secured to receiver 32 in a manner to co-rotate. In the illustrated embodiment, first body 40 may be secured to receiver 32 so as to be co-rotatable about first axis 66. Catheter body 18 may be secured to receiver 32 to correspondingly co-rotate with first body 40. It is to be understood, however, that catheter body 12 may otherwise be secured to second body 50 so as to be co-rotatable therewith relative to first body 40.

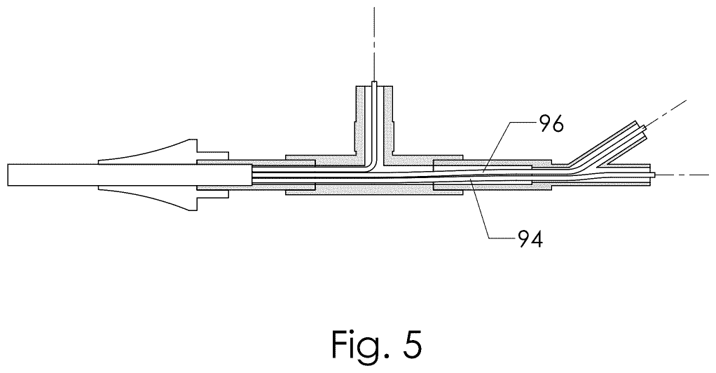

As illustrated in FIG. 5, tubes may be employed to fluidically connect one or more of ports 42, 44, 52 and respective lumens 18. In some embodiments, a first tube 94 may be arranged to fluidically connect first port 42 and first lumen 18a through first channel 70. A second tube 96 may be arranged to fluidically connect second port 44 and second lumen 18b through stem channel 90 and first channel 70. One or more additional tubes (not shown) may be arranged to fluidically connect port 52 to one or more respective lumens 18. In some embodiments, such additional tubes may extend through second channel 80, and may further extend through at least a portion of first channel 70. For the purposes hereof, the term “fluidically connect” is not intended to be limited to the conveyance of fluid, but rather to refer to the establishment of a continuous passage from one point to another. Thus, tubes, such as first and second tubes 94, 96 may be employed to convey fluids, procedural instruments, guidewires, steering cables, and the like.

An operational advantage introduced by control handle 30 of the present invention is the ability to co-rotate catheter body 12 with one or more fluidically connected tubes or channels about an axis while maintaining another tube or channel relatively stationary. Doing so limits or prevents tangling or kinking of tubing lines, and also permits one or more fluid delivery pathways to be relatively static.

In an example embodiment of a thrombectomy catheter, control handle 30 may be arranged to accommodate a guide wire through first channel 70 to a respective lumen 18 within catheter body 12. In some embodiments, the guide wire is passable through first port 42 and first tube 94 to and through first lumen 18a. A first fluid, such as a pressurized process fluid may be conveyed through one of second port 44 and port 52 into communication with a respective lumen 18. In some embodiments, the pressurized process fluid may be delivered from a pressurized fluid source through one of second port 44 and port 52 and through a respective lumen 18, such as second lumen 18b to distal end 14 of catheter body 12 to perform a hydrodynamic thrombectomy procedure at a target body location. Return fluid may be conveyed through a respective different lumen 18, such as third lumen 18c, and then through the other of second port 44 and port 52 to disposal. In some embodiments, the pressurized process fluid may be driven through second tube 96 through first channel 70, and the return fluid may be conveyed through first channel and second channel to port 52. In such an embodiment, the return fluid may be prevented from exiting manifold apparatus 60 by one or more sealed ends (not shown) at one or more of first channel 70 and stem channel 90.

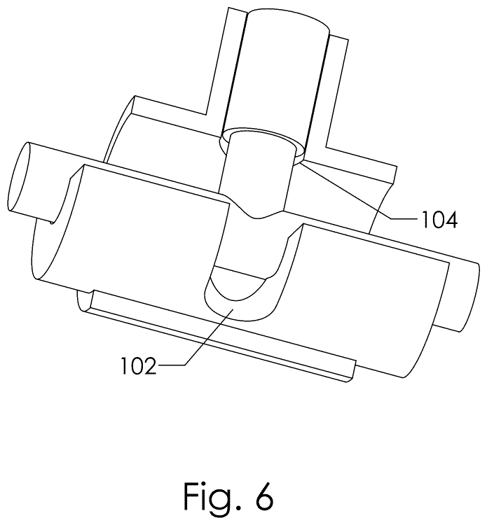

Control handle 30 may further include structure for limiting relative rotation of first portion 40 with respect to second portion 50. Such limitation can aid in preventing undesired tangling and/or kinking of tubing within control handle 30. In the embodiment illustrated in FIG. 6, first portion 40 of housing 36 includes a rotation control element 102, and second portion 50 of housing 36 includes a rotation limiter 104. Rotation control element 102 and rotation limiter 104 are mutually arranged such that rotation control element 102 contacts rotation limiter 104 upon a threshold extent of relative rotation about first axis 66. The contact between rotation control element 102 and rotation limiter 104 preferably arrests further relative rotation among first and second portions 40, 50 of housing 36. In some embodiments, rotation control element 102 and rotation limiter 104 are arranged to arrest relative rotation of first and second portions 40, 50 at no more than 330° C. rotation about first axis 66. In some embodiments, rotation control element 102 and rotation limiter 104 are arranged to arrest relative rotation of first and second portions 40, 50 at no more than 300° C. rotation about first axis 66. In some embodiments, rotation control element 102 and rotation limiter 104 are arranged to arrest relative rotation of first and second portions 40, 50 at no more than 270° C. rotation about first axis 66.

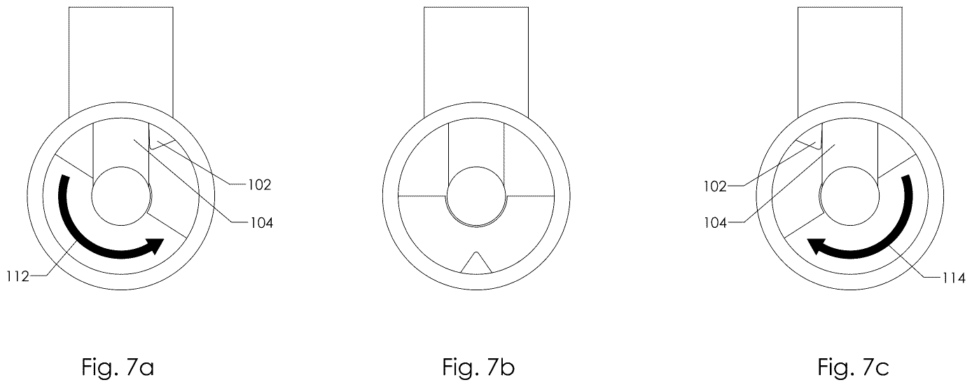

FIGS. 7A-7C illustrate the rotation control provided by rotation control element 102 and rotation limiter 104. In particular, FIGS. 7A-7C illustrate a rotational sequence from a maximum first rotational direction 112 in FIG. 7A to a maximum second rotational direction 114 in FIG. 7C. FIG. 7B illustrates a rotational mid-point or neutral relative position among first and second portions 40, 50 of housing 36. Rotation control element 102 is illustrated in FIG. 7A in a relative rotational position in contact with rotation limiter 104 to arrest further relative rotation along first rotational direction 112. Rotation control element 102 is illustrated in FIG. 7B in a relative rotational position out of contact with rotation limiter 104, such that relative rotation among first and second portions 40, 50 is not resisted or arrested by a relationship between rotation control element 102 and rotation limiter 104. Rotation control element 102 is illustrated in FIG. 7C in a relative rotational position in contact with rotation limiter 104 to arrest further relative rotation along second rotational direction 114. Although the embodiment illustrated in FIGS. 7A-7C includes a single rotation control element 102 and a single rotation limiter 104, it is contemplated that control handle 30 may include one or more of each of rotation control element 102 and rotation limiter 104.

In some embodiments, one or more regions of housing 36 of control handle 30 may include an exterior surface texture or material that exhibits high friction to facilitate operator grip upon control handle 30. High friction surface 120 may be disposed in one or more regions of an exterior surface housing 36 that constitute less than an entirety of the exterior surface, or may cover an entirety of the exterior surface of housing 36. In one embodiment, high friction surface may be disposed at only one of first and second portions 40, 50 of housing 36.

In some embodiments, one or more regions of housing 36 of control handle 30 may include markings, indicia, and/or structural features that indicate an extent of relative rotation of first and second portions about first axis 66. In some embodiments, both of first and second portions of housing 36 include such markings, indicia, and/or structural features to indicate relative rotation thereof.

The invention has been described herein in considerable detail in order to provide those skilled in the art with information needed to apply the novel principles of the present invention. However, it is to be understood that various modifications may be made without departing from the scope of the invention.

1. A directional control handle for a multiple-lumen catheter device, the handle comprising:
a receiver for coupling to a proximal end of the catheter device;
a first portion defining an axial channel along an axis, the first portion including a first port fluidically connected to the proximal end of the catheter device through the axial channel;
a second portion including a second port, and defining a second channel in fluid communication with and extending non-axially from the axial channel for fluidically connecting the second port to the proximal end of the catheter device, the first portion being rotatable about the axis relative to the second portion; and
a third port associated with one of the first and second portions, and being fluidically connected to the proximal end of the catheter device through at least one of the axial channel and the second channel.

2. The directional control handle as in claim 1 wherein the second port is fluidically connected to the proximal end of the catheter device through the second channel and the axial channel.

3. The directional control handle as in claim 1 wherein the third port is fluidically connected to the proximal end of the catheter device through only the axial channel.

4. The directional control handle as in claim 3, including a first tube extending between the first port and a first lumen of the catheter device, and a second tube extending between the third port and a second lumen of the catheter device.

5. A multiple-lumen catheter apparatus, comprising:
a catheter body having a distal end, a proximal end, a longitudinal axis extending centrally within the catheter body between the proximal end and the distal end, and a plurality of distinct lumens extending between the proximal and distal ends;
a directional control handle connected to the proximal end of the catheter body, the directional control handle including:
(i) a housing having a first portion and a second portion;
(ii) a manifold apparatus adapted to connect to the proximal end of the catheter body, the manifold apparatus including a junction member and a first connector rotatably connected to the junction member about a first axis, the first connector defining at least a portion of a first channel along the first axis for fluidically connecting a first port of the directional control handle to the proximal end of the catheter body, and the junction member defining a second channel along a second axis for fluidically connecting a second port of the directional control handle to the proximal end of the catheter body; and
(iii) a third port of the directional control handle arranged for fluidic connection to the proximal end of the catheter body through at least one of the first and second channels,
wherein the first portion of the housing is fixedly secured to the first connector and rotatable relative to the second portion of the housing about the first axis.

6. The catheter apparatus as in claim 5 wherein the junction member includes a first leg and a second leg, with the first connector being rotatably connected to the first leg.

7. The catheter apparatus as in claim 6, including a second connector rotatably connected to the second leg of the junction member about the first axis, the second connector defining a portion of the first channel.

8. The catheter apparatus as in claim 7 wherein the second channel intersects the first channel at a junction.

9. The catheter apparatus as in claim 8 wherein the first and third ports are fluidically connected to the first channel through the second connector.

10. The catheter apparatus as in claim 9, including a first tube fluidically connecting the first port to a first lumen of the catheter body.

11. The catheter apparatus as in claim 10, including a second tube fluidically connecting the third port to a second lumen of the catheter body.

12. The catheter apparatus as in claim 11 wherein a third lumen of the catheter body is fluidically connected to the second port through the second channel.

13. The catheter apparatus as in claim 12 wherein the second connector is fixedly connected to the first portion of the housing.

14. The catheter apparatus as in claim 13 wherein the junction member is fixedly connected to the second portion of the housing.

15. The catheter apparatus as in claim 14 wherein the portion of the first channel defined by the second connector is fluidically disconnected from the second channel.

16. The catheter apparatus as in claim 5, wherein the first portion of the housing includes a rotation control element that is contactable with a rotation limiter of the second portion for limiting relative rotation of the first and second housing portions.

17. The catheter apparatus as in claim 5 wherein rotation of the first connector about the first axis correspondingly rotates the proximal end of the catheter body about the first axis.

18. A method for controlling a catheter device having a catheter body with a distal end, a proximal end, a longitudinal axis extending centrally within the catheter body between the proximal end and the distal end, a plurality of distinct lumens extending between the proximal and distal ends, and a control handle secured to the proximal end, wherein the control handle defines at least two fluid channels, with a first fluid channel formed along a first axis through a first portion of the control handle and fluidically connected to a first lumen of the catheter body, and a second fluid channel formed along a second axis through a second portion of the control handle and fluidically connected to a second lumen of the catheter body, the second axis being non-parallel to the first axis, the method comprising:
rotating the first portion of the control handle with respect to the second portion of the control handle about the first axis to correspondingly rotate the catheter body about the longitudinal axis, wherein the first and second axes are capable of being mutually stationary during the rotation.

19. The method as in claim 18 wherein rotation of the first portion of control handle with respect to the second portion of the control handle about the first axis correspondingly rotates the catheter body with respect to the second fluid channel.
